(19) United States
(12) Patent Application Publication
(10) Pub. No.: US 2024/0358246 A1
(43) Pub. Date: Oct. 31, 2024

(54) ENDOSCOPE REPROCESSING SYSTEM AND TECHNIQUE TO DETECT MISCONNECTION OF A ROTATING SPRAY ASSEMBLY

(71) Applicant: Ecolab USA Inc., Saint Paul, MN (US)

(72) Inventors: Sébastien RUIZ, Saint-Victoret (FR); Eric PENDARIES, La Ciotat (FR)

(21) Appl. No.: 18/646,036

(22) Filed: Apr. 25, 2024

Related U.S. Application Data
(60) Provisional application No. 63/499,075, filed on Apr. 28, 2023.

(51) Int. Cl. A61B 1/12; A61L 2/22

(52) U.S. Cl. CPC A61B 1/123; A61L 2/22; A61L 2202/122; A61L 2202/14; A61L 2202/24

(57) ABSTRACT

Medical device reprocessing systems, such as endoscope reprocessing systems, can include a processing chamber configured to receive one or more medical devices to be cleaned and a spray arm assembly configured to rotate relative to processing chamber. The spray arm assembly can include a spray arm and a passive emitter. A non-contact receiver coupled to the processing chamber can detect the presence of the passive emitter, and a controller in communication with the non-contact receiver can detect when the passive emitter is positioned at the detectable position proximate the non-contact receiver and increment a count of detected rotations of the spray arm assembly each time the passive emitter is positioned at the detectable position proximate the non-contact receiver and compare the count to an expected count. If the count is lower than expected, the controller can output an alert or shut down the system.

### RELATED APPLICATION

This application claims priority to U.S. Provisional Application No. 63/499,075, filed on Apr. 28, 2023, the entire contents of which are incorporated herein by reference.

### TECHNICAL FIELD

This disclosure relates to systems and techniques for reprocessing reusable medical equipment and, more particularly, systems and techniques for reprocessing endoscopes.

### BACKGROUND

An endoscope is a slender, tubular optical instrument used as a viewing system for examining an inner part of the body and, when used with an attached instrument, for biopsy or surgery. Decontamination systems can be used to reprocess previously-used medical devices, such as endoscopes such that the devices can be used again on a subsequent patient. During the decontamination process of an endoscope, the endoscope can be inserted into a chamber of a reprocessing machine and the internal channels of the endoscope connected to the machine to receive cleaning and/or disinfecting agents. For example, the reprocessing machine may provide a system of lines, pumps and valves for the purpose of feeding a cleaning and/or disinfecting agent to the endoscope placed in a chamber. The external surface of the endoscope may be sprayed with cleaning and/or disinfecting agent delivered through one or more spray assemblies inside of the chamber in which the endoscope is being reprocessed.

In practice, the reprocessing machine itself may be periodically disassembled and cleaned to ensure the internal sterility of the machine. For example, one or more spray assemblies inside of the reprocessing machine may be detached and the internal surfaces of the chamber cleaned and/or sterilized. The spray assemblies may be separately cleaned and/or sterilized and reinstalled inside of the chamber of the reprocessing machine. If the spray assemblies are not appropriately reinstalled, fluid intended to be sprayed through the assemblies during subsequent operation of the reprocessing machine may instead leak at the connection. This can undermine the performance of the reprocessing machine.

### SUMMARY

In general, this disclosure is directed to devices, systems, and techniques for reprocessing medical instruments such as endoscopes and other reusable medical instruments. In some examples, the disclosure describes devices, systems, and techniques for determining the adequacy of a flow of fluid within the reprocessing system, such as through one or more rotating spay assemblies in the system.

Reprocessing systems can include one or more spray arm assemblies configured to spray fluid within a processing chamber. Spray arm assemblies can be configured to rotate relative to a processing chamber, for example, as a result of fluid being applied thereto such that the rotational speed of the spray arm assembly is related to the amount of fluid provided to the spray arm assembly.

Defects in the fluid line, such as clogs, an improperly connected spray arm assembly, or a malfunctioning fluid pump, can change the flow of fluid through the spray arm assembly, and therefore the rotational speed of the spray arm assembly. Physical obstructions to the spray arm assembly can also affect the rotation of the spray arm assembly and the distribution of fluid throughout the processing chamber.

A spray arm assembly can include a passive emitter, such as a magnet that emits a magnetic field, included therewith such that, as the spray arm assembly rotates, the passive emitter revolves around the rotational axis of the spray arm assembly. The processing chamber can include a non-contact receiver, such as a magnet sensor, that can detect when the passive emitter of the spray arm assembly moves to a detectable position while the spray arm assembly rotates. In some examples, each time the passive emitter reaches the detectable position, such that it can be detected by the non-contact receiver, the spray arm assembly has completed a single revolution.

A controller can receive a signal from the non-contact receiver and count the number of revolutions of the spray arm assembly. For example, the controller can be configured to detect when the passive emitter is positioned at the detectable position, and each time the passive emitter is positioned at the detectable position, increment a count of detected rotations of the spray arm assembly.

The controller can compare the count of detected rotations of the spray arm assembly to an expected number of rotations. For example, the controller can determine whether the count of rotations exceeds a minimum number of expected rotations. In some cases, a number of detected rotations below the expected number indicates a defect in the fluid line, such as a clog, an improperly connected spray arm assembly, or a malfunctioning pump. Additionally or alternatively, a number of detected rotations below the expected number can indicate an obstruction to the rotation of the spray arm assembly, such as one or more medical devices to be cleaned obstructing the rotational path of the spray arm assembly. The controller can be configured to output an alert, such as via a user interface, if the count of detected rotations is below an expected number of detected rotations. Additionally or alternatively, in some examples, if the count of detected rotations is below an expected number of detected rotations, the controller can stop the operation of a reprocessing procedure.

The alert can inform a user that the system is not operating properly, and the user can then inspect the system before performing a full reprocessing procedure. Otherwise, if left uncorrected, improper fluid flow during the procedure can lead to unsatisfactorily cleaned and/or sanitized components, which can be dangerous for subsequent use. A user receiving the alert can inspect for one or more fluid flow errors, such as one or more clogs, an improperly connected spray arm assembly, or a malfunctioning pump, and/or can inspect for an obstruction to the rotation of the spray arm assembly, such as one or more medical devices impeding rotation. Once one or more issues has been addressed, system can be started up and the controller can monitor the rotation of the spray arm assembly to confirm that the spray arm assembly is rotating as expected. However, if the problem has not been adequately addressed, the controller will continue to detect an error in system operation based on the rotation of the spray arm assembly and may output another alert and/or continue to prevent system operation.

In an example implementation, a spray arm assembly is configured to engage a stator portion of the processing chamber such that the spray arm assembly rotates relative thereto. A magnet is positioned within a rotating hub of the spray arm assembly. During operation, fluid is provided to the spray arm assembly and directed to one or more spray arms connected to the hub, causing the spray arm assembly to rotate and the magnet to revolve around the axis of rotation of the spray arm assembly and, during each revolution, the magnet passes through a detectable position over a magnet sensor held by the stator portion. A controller receives a signal from the magnet sensor that indicates when the magnet travels through the detectable position. The controller can count the number of revolutions of the spray arm assembly and compare the number of revolutions to an expected number such as described herein.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a block diagram of one example configuration of an automated endoscope reprocessing machine (AER).

FIG. 2 shows an example processing chamber of the example AER machine of FIG. 1.

FIG. 3 shows the medical device reprocessing system of FIG. 2 with some components removed for easier visualization.

FIG. 4 shows a perspective view of a spray arm assembly attached to a stator portion of a processing chamber.

FIG. 5. shows a top-down view of the spray arm assembly engaging the stator portion of the processing chamber.

FIG. 6 shows an exploded view of a spray arm assembly and stator portion.

FIGS. 7A-FE show example operation of a non-contact receiver outputting a pulse signal representing the presence of a passive emitter within a detectable position.

FIG. 8 shows a cross-sectional view of an example embodiment including a spray arm assembly including a spray arm extending parallel to a rotational axis and normal to the processing chamber floor.

FIG. 9 shows a perspective view of an example reprocessing system including a processing chamber and a plurality of spray arm assemblies.

FIG. 10 is a process flow diagram showing an example operation of a controller determining whether the system is operating properly.

### DETAILED DESCRIPTION

This disclosure generally relates to endoscope reprocessing machines and related endoscope reprocessing techniques for processing an endoscope previously inserted into and used on a patient to be suitable for reuse on a subsequent patient. In some examples, the disclosed systems and techniques are implemented to evaluate the quality of a connection made between the endoscope reprocessing machine and a rotatable spray assembly within a processing chamber of the machine.

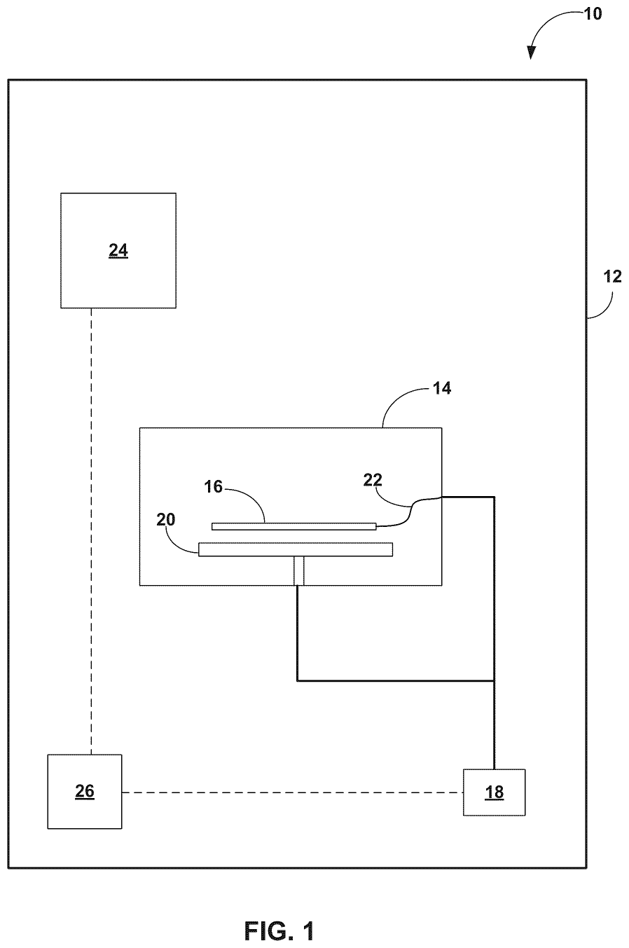

An automated endoscope reprocessing machine (AER) implementing the concepts and techniques of the disclosure can have a variety of different features and configurations. FIG. 1 is a block diagram of one example configuration of an AER that can detect connectivity of an endoscope biopsy channel to the machine according to the disclosure. In the example of FIG. 1, an AER 10 is shown in that includes an exterior housing 12 arranged to contain and provide protection for the components of the AER. AER 10 includes one or more processing chambers 14 configured to receive a medical device 16 to be processed therein, such as an endoscope 16. AER 10 also includes one or more doors to provide access to each processing chamber 14 for loading endoscope 16 in the chamber, sealing the chamber during processing, and for removing the endoscope from the processing chamber after processing. In different implementations, the AER 10 can be configured as a basin-style machine in which endoscope 16 is loaded into and unloaded from processing chamber 14 through a top or upper opening or as a pass-through style machine in which endoscope 16 is loaded through a first door one side of the machine into processing chamber 14 (e.g., a dirty or non-sterile side) and unloaded from the processing chamber through a second door on an opposite side of the machine (e.g., a clean or sterile side).

In use, a clinician may place endoscope 16 into an endoscope carrier that is then positioned in processing chamber 14. The endoscope carrier may be a basket have a wire frame or lattice structure that allows ingress and egress of fluids into and out of the carrier once positioned inside of processing chamber 14. In some examples, the clinician can fluidly connect each of the channels of endoscope 16 to a connector on the endoscope carrier. The connector carried by the endoscope carrier can then be mechanically and/or fluidly connected to a corresponding connector within processing chamber 14 to fluidly couple the individual channels to AER 10. This can simplify the process of connecting endoscope 16 to AER 10 rather than inserting the endoscope into processing chamber 14 and then connecting each individual channel of the endoscope to the machine within the processing chamber.

AER 10 can include a circulation system that can circulate one or more reprocessing fluids 18 such as detergent, sterilant, disinfectant, water, alcohol, air, and/or any other suitable fluid, for example, through endoscope 16, partially or fully immerse the endoscope in the fluid within processing chamber 14, and/or spray the fluid onto the exterior surface of the endoscope. The circulation system can include a fluid supply and a circulation pump, where the circulation pump can be fluidly connected to the fluid supply such that the fluid can be drawn from the fluid supply into the circulation system. In certain implementations, the circulation system can include a mixing chamber in which the fluid can be mixed with another fluid, such as water, for example, to form a mixed or dilute fluid for delivery to processing chamber 14 and endoscope 16 therein.

In either event, processing chamber 14 can include one or more spray nozzles 20 which can be in fluid communication with the one or more fluid 18, e.g., via a circulation pump such that the fluid pressurized by the circulation pump can be ejected from the circulation system through the spray nozzles and onto an exterior surface of endoscope 16. In some examples, each processing chamber 14 includes a plurality of spray nozzles 20 positioned around the perimeter thereof and/or one or more spray nozzles which can spray upwardly from the floor of the processing chamber.

For example, in some implementations, processing chamber 14 includes one or more rotating arm members. The rotating arm members can be rotatably mounted via a central hub sleeve rotatably connected around a rotating arm hub. Each spray arm can define a spray arm lumen. The spray arm lumens can extend at least a portion of the length of the spray arm and serve to operatively connect a hub sleeve lumen defined within the central hub sleeve with a plurality of spray jets. Together the interconnected hub sleeve lumen, spray arm lumens, and outlet spray openings can provide a conduit for discharging pressurized fluids.

In order to clean, disinfect, and/or sterilize internal channels within endoscope 16, AER 10 can include one or more supply lines 22 in fluid communication with the one or more fluids 18 (e.g., via a circulation system pump) that can be placed in fluid communication with the internal channels and/or ports of the endoscope. In some examples, processing chamber 14 can include one or more complementary connectors that comprise the ends of the supply lines. The individual channels and/or ports of endoscope 16 can be fluidly connected to a master connector which, in term, is connected to the complementary connector inside of AER 10. In some examples, AER 10 can further include one or more flexible conduits which can be connected and/or sealingly engaged with the ports and/or the channels defined by endoscope 16 such that the pressurized fluid from AER 10 can fluid into the individual channels of the endoscope via the flexible conduits. A variety of different connector configurations can be used to make a mechanical and/or fluid connection between AER 10 (e.g., a fluidly conduit in fluid communication with the machine) and the individual channels and/or ports of endoscope 16, such as threaded connectors, bayonet connectors, cam and groove connectors, and the like. In either case, each of the one or more connections made by the clinician between AER 10 and the individual channels and/or ports of endoscope 16 is desirably fluid tight to prevent fluid intended to be passed through the corresponding channel of the endoscope from bypassing at the connection location.

In operation, a clinician may perform one or more manual reprocessing steps on endoscope 16 prior to introducing the endoscope in AER 10. The type of manual cleaning activities may be performed on the endoscope include disassembly and removal of components, applying brushes to clear channels, wiping to remove visible liquids and solids, and other human-performed cleaning actions. After completion of any desired manual reprocessing steps, the clinician can introduce endoscope 16 into processing chamber 14 of AER 10 for automated reprocessing. In some examples, the clinician may insert endoscope 16 into an endoscope carrier and fluidly connect one or more (e.g., optionally all) of the individual channels of the endoscope to a multi-port connector carried by the carrier. For example, the clinician may fluidly connect the inlet of a biopsy channel of endoscope 16 and the inlet of a suction channel of the endoscope to the multi-port connector carried by the carrier. The clinician can then insert the carrier into processing chamber 14, with the multi-port connector mechanically and/or fluidly connecting to a corresponding connector within processing chamber 14. Additionally or alternatively, the clinician can insert endoscope 16 into processing chamber 14 and connect one or more (e.g., optionally all) of the individual channels of the endoscope, including the inlet of a biopsy channel and the inlet of a suction channel, to corresponding connection lines of AER 10 to place the channels in fluid communication with the machine.

AER 10 can include a user interface 24 that a clinician can interact with to input information for controlling the AER and/or to output information back to the clinician. User interface 24 may be implemented using a presence-sensitive display, such as a resistive touchscreen, a surface acoustic wave touchscreen, a capacitive touchscreen, a projective capacitance touchscreen, a pressure sensitive screen, an acoustic pulse recognition touchscreen, or another presence-sensitive display technology. User interface 24 may function as an output (e.g., display) device using any one or more display devices, such as a liquid crystal display (LCD), dot matrix display, light emitting diode (LED) display, organic light-emitting diode (OLED) display, or similar display capable of outputting visible information to the user. User interface 24 may include physically-depressible buttons that may receive tactile input from a clinician using AER 10.

AER 10 can include one or more controller 26 that manage the overall operation of the AER. Controller 26 can be communicatively coupled to sensors, supply control devices (e.g., pumps, valves), and/or other controllable components of AER 10 to manage the overall operation of the machine. Controller 26 includes a processor and a memory. The memory can store software for running the controller and may also store data generated or received by the processor. The processor can run software stored in the memory to manage the operation of the device.

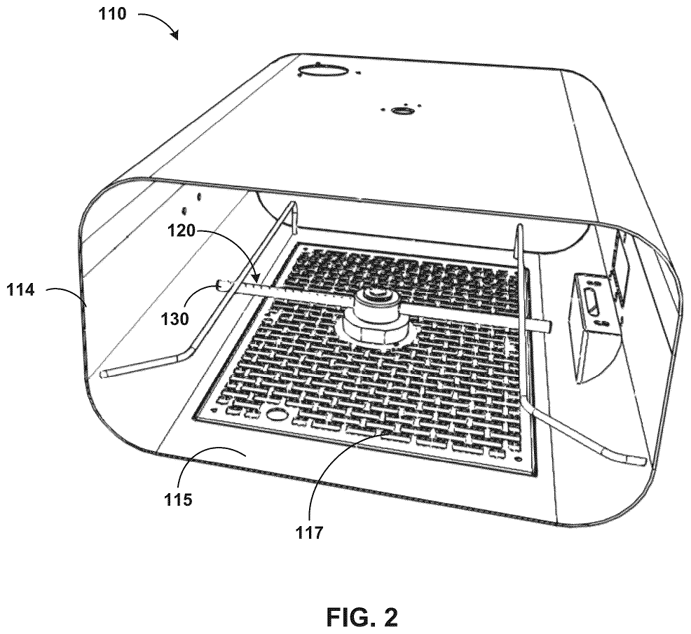

FIG. 2 shows an example configuration of a processing chamber that can be used on AER 10. In some examples, the reprocessing system 110 is configured for receiving and reprocessing endoscopes although can be used for receiving and processing other reprocessable instruments. In particular, the illustrated system includes a processing chamber 114 configured to receive one or more medical devices to be cleaned, such as, in some embodiments, one or more endoscopes. While shown as having open sides for ease of visualization and explanation, processing chamber 114 is configured to be closed to form a closed volume inside which one or more medical devices can be reprocessed.

The system further includes a spray arm assembly 120 disposed within the processing chamber 114. In some embodiments, the spray arm assembly 120 comprises at least one spray arm 130 configured to expel a fluid outward toward one or more medical devices within the processing chamber 114.

In the illustrated example, spray arm assembly 120 is coupled to a floor 115 of the processing chamber 114, though in other examples, spray arm assembly 120 can be coupled to any position within the processing chamber 114. The floor 115 of the processing chamber 114 further includes a grate 117 that can be configured to allow fluid used in a reprocessing process, such as fluid expelled from the spray arm assembly, to be drained from the inside of the processing chamber 114.

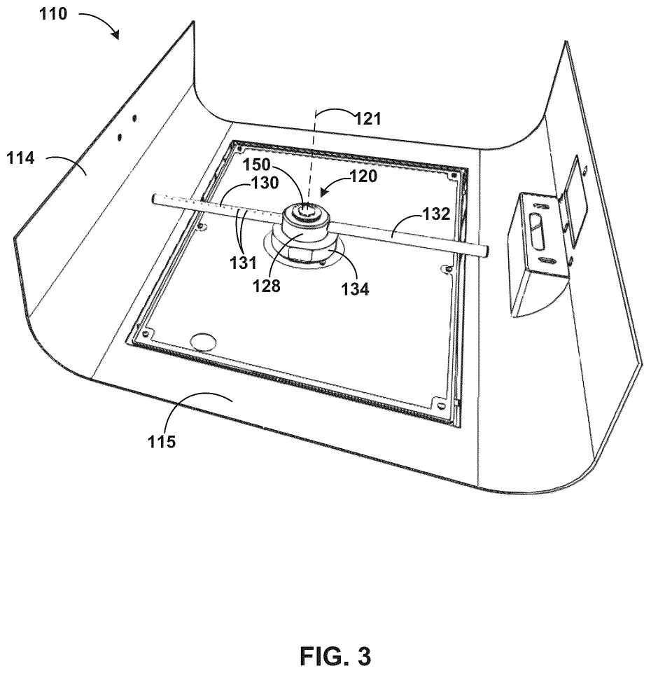

FIG. 3 shows the medical device reprocessing system of FIG. 2 with some components removed for easier visualization.

In some embodiments, the spray arm assembly 120 is configured to rotate about a rotational axis 121 relative to the processing chamber 114, such as relative to the floor 115 of the processing chamber 114. In some examples, the rotational axis 121 is normal to the floor 115 of the processing chamber 114 and extends through the center of the spray arm assembly 120.

In some embodiments, the spray arm assembly 120 is removably attached to the processing chamber 114. In an example configuration, spray arm assembly 120 is secured to the processing chamber via cap 150. In some embodiments, cap 150 does not rotate with the spray arm assembly 120 as it rotates about the rotational axis 121. In other examples, cap 150 rotates with the spray arm assembly 120 as it rotates about the rotational axis 121. In various examples, cap 150 can be threadably engaged with the processing chamber 114 and/or with the spray arm assembly 120. For instance, in some examples, cap 150 can include a bolt configured to extend through a portion of the spray arm assembly 120 and thread into a portion of the processing chamber 114. Additionally or alternatively, in some examples, cap 150 can be press fit into one or both of the spray arm assembly 120 and the processing chamber 114. Other attachment mechanisms are possible and within the scope of this disclosure, including one or more springs, clips, or other attachment mechanisms. In some embodiments, cap 150 renders the spray arm assembly 120 removably attached to the processing chamber 114, for example, permitting the spray arm assembly 120 to be removed from processing chamber 114 when cap 150 is removed from the spray arm assembly 120 and the processing chamber 114. In some examples, spray arm assembly 120 is configured to removably engage processing chamber 114 without cap.

The spray arm assembly 120 in FIGS. 2 and 3 includes a first spray arm 130 extending from a hub 128 parallel to the floor 115 of the processing chamber 114 and a second spray arm 132 extending from the hub 128 parallel to the floor 115 of the processing chamber 114 in an opposite direction as the first spray arm 130. In some embodiments, first spray arm 130 and second spray arm 132 extend in opposite directions along a spray arm axis. As shown in the illustrated examples of FIGS. 2 and 3, the first spray arm 130 comprises a plurality of spray holes 131 positioned along the first spray arm 130 and separated from one another along the direction of the spray arm axis.

In some embodiments, spray arm assembly 120 is configured to receive a fluid from the processing chamber 114. In various examples, receiving fluid from the processing chamber can include receiving fluid from one or more vessels, such as one or more tubes or hoses, connected to the processing chamber 114 and configured to provide fluid thereto from a fluid source, such as a reservoir. One or more pumps can be used to provide such fluid to the processing chamber 114. Fluid can include a reprocessing fluid, such as a cleaning fluid, a sanitizing fluid, a rinsing fluid, or other fluids for use in a reprocessing procedure. In some embodiments, spray arm assembly 120 receives fluid from the processing chamber 114 and directs the fluid to one or more spray arms (e.g., 130, 132). The one or more spray arms (e.g., 130, 132) can be configured to expel the fluid through one or more spray holes (e.g., 131) therein.

Various spray hole configurations can be used. For example, a spray arm can include any number of spray holes, and spray holes can be any shape. In some examples, a spray arm can include spray holes having different shapes and sizes. In some examples, a spray arm assembly includes between 10 spray holes and 40 spray holes. In some examples, spray holes are distributed between a plurality of spray arms, such as 10 spray holes positioned on each of two spray arms for 20 total spray holes, or 16 spray holes positioned on each of two spray arms for a total of 32 spray holes. In some examples, spray holes are circular, and in some examples, spray holes are circular and have a diameter of between 1 and 2 mm. In some embodiments, spray holes can be other shapes, such as oblong shapes. In an example embodiment, a spray arm assembly includes a plurality of oblong spray holes and a plurality of circular spray holes.

In some examples fluid is provided to the spray arm assembly 120 from the processing chamber 114 at a flow rate or within a range of flow rates. In some embodiments, fluid is provided at between approximately 0.5 and 0.6 bar gauge pressure and flows at between approximately 150 and 250 liters per minute. In other examples, fluid is provided at between approximately 1.7 and 2.5 bar gauge pressure and flows at between 20 and 30 liters per minute.

In some embodiments, the rate of rotation of the spray arm assembly 120 is associated with the flow rate and/or pressure of the fluid. For instance, in some embodiments, expelling the fluid from the spray arm 130 causes the spray arm assembly 120 to rotate about the rotational axis 121. Thus, in some embodiments, during operation of the reprocessing system 110, fluid is provided to the spray arm assembly 120 and expelled through one or more spray holes (e.g., 131) in a spray arm (e.g., 130). The fluid causes the spray arm assembly 120 to rotate such that fluid is expelled in multiple directions throughout reprocessing. In some embodiments, cap 150 includes one or more spray holes such that fluid provided to the spray arm assembly 120 is also expelled through cap 150. In various such embodiments, the cap 150 can rotate with the spray arm assembly 120 or remain fixed relative to the processing chamber 114 while the spray arm assembly 120 rotates.

As shown, spray arm assembly 120 is coupled to a floor 115 of processing chamber 114. Spray arm assembly 120 can be configured to rotate about a rotational axis 121 relative to the processing chamber 114. Fluid provided to the spray arm assembly 120 can cause rotation of the spray arm assembly 120 while fluid is expelled from spray holes (e.g., 131) in one or more spray arms (e.g., 130) of the spray arm assembly 120.

In some embodiments, the processing chamber 114 includes a stator portion 134 configured to engage the spray arm assembly 120 wherein the spray arm assembly 120 rotates relative to the stator portion 134. In some examples, spray arm assembly 120 is attached to the stator portion 134, such as via cap 150. In other examples, the spray arm assembly engages the stator portion 134 by being positioned on or above the stator portion 134 without being attached thereto. In some embodiments, the stator portion 134 is configured to provide fluid to the spray arm assembly 120 during operation of the reprocessing system. In some embodiments, the stator portion 134 comprises a fitting designed to engage a corresponding fitting of the spray arm assembly 120.

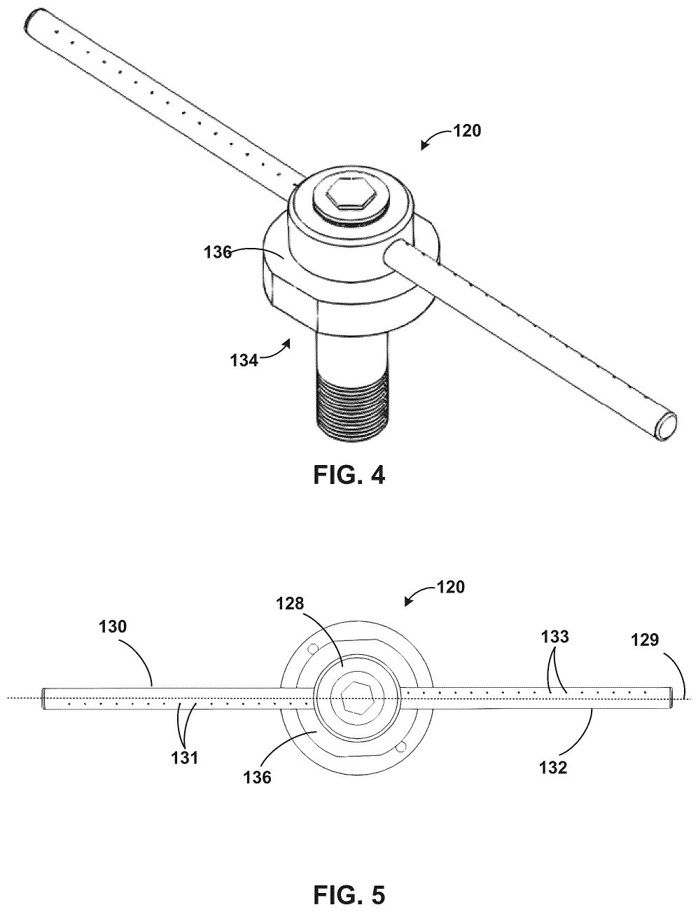

FIG. 4 shows a perspective view of a spray arm assembly 120 attached to a stator portion 134 of a processing chamber. In some embodiments, stator portion 134 comprises a stage 136, and spray arm assembly 120 engages the stage 136. Spray arm assembly 120 can rotate relative to stage 136 as fluid is provided thereto.

FIG. 5. shows a top-down view of the spray arm assembly engaging the stator portion of the processing chamber. As shown, spray arm assembly 120 including spray arms 130, 132, is positioned on top of stage 136 of stator portion 134. Spray arms 130, 132 extend from hub 128 along a spray arm axis 129. As shown in the illustrated example, first spray arm 130 includes a plurality of spray holes 131 separated from one another along the direction of the spray arm axis 129 and positioned on a first side of the spray arm axis 129. Second spray arm 132 includes a plurality of spray holes 133 separated from one another along the direction of the spray arm axis 129 and positioned on a second side of the spray arm axis 129.

In some embodiments, during operation, fluid expelled from spray holes 131 of the first spray arm 130 cause the spray arm assembly 120 to rotate in a clockwise direction relative to the view in FIG. 5. Similarly, in some embodiments, fluid expelled from spray holes 133 of the second spray arm 32 also cause the spray arm assembly 120 to rotate in the same clockwise direction. Thus, in some embodiments, a spray arm assembly includes a plurality of spray arms, each comprising spray holes configured such that, when fluid is expelled from the spray holes each of the spray arms cause the spray arm assembly to rotate in the same direction.

In some cases, reprocessing one or more medical devices involves providing a minimum amount of fluid to the medical devices within the processing chamber. Less fluid than prescribed can lead to improper or incomplete reprocessing phases resulting in unclean medical devices. This can occur, for example, if a fluid pump is operating outside of a normal or expected operating range. Similarly, one or more clogs in a spray arm assembly (e.g., in a hub and/or one or more spray holes) can prevent fluid from reaching areas that are otherwise intended to receive fluid to for cleaning or sanitizing. Poor engagement of a spray arm assembly into the processing chamber can lead to poor fluid coupling and direction of fluid to the intended destination. Additionally or alternatively, in some cases, physical obstruction to the rotation of the spray arm assembly can affect the distribution of fluid throughout the processing chamber, such as be preventing fluid from being provided to one or more intended locations.

In some embodiments, one or more sensors can be used to monitor the rotation of the spray arm assembly during operation to confirm that the spray arm assembly is rotating as expected. If so, it increases the likelihood that the spray arm assembly is present, is properly coupled to the processing chamber, is not clogged, that a pump delivering the fluid is operating as expected, and that there are no physical obstructions to the rotation of the spray arm assembly.

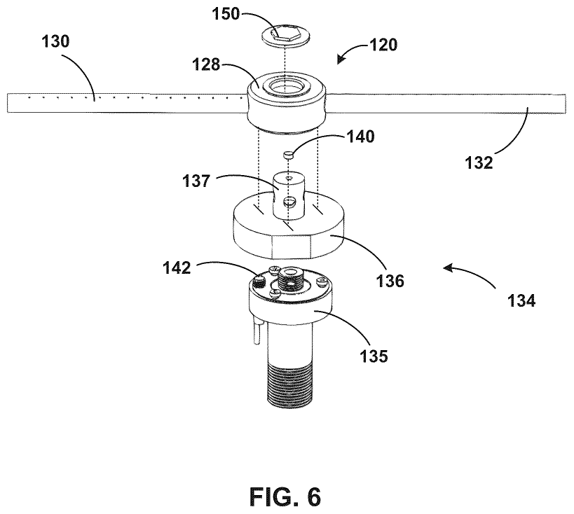

In some examples, the spray arm assembly comprises a passive emitter that is detectable by a non-contact receiver positioned in the stator. FIG. 6 shows an exploded view of a spray arm assembly and stator portion. In the illustrated example, spray arm assembly 120 includes a hub 128 and spray arms 130, 132 extending therefrom. Stator portion 134 includes a base 135 and a stage 136 configured to sit on top of the base and receive the spray arm assembly 120. A cap 150 can secure the spray arm assembly 120 to the stage 136.

In the illustrated example, the stage 136 includes a post 137 onto which the hub 128 of the spray arm assembly 120 can set. The post 137 includes a plurality of holes therein that can direct fluid received from the processing chamber into the spray arms 130, 132 of the spray arm assembly 120.

Spray arm assembly 120 includes a passive emitter 140 configured to be positioned inside the hub 128 proximate the stage 136. The passive emitter 140 is located away from the rotational axis such that, as the spray arm assembly 120 rotates, the passive emitter 140 traverses a circle around the rotational axis. In various examples, the passive emitter can include a magnet, an inductively detectable (e.g., a metallic) component, an optically detectable component, or a capacitively detectable component.

The base 135 of the stator portion 134 includes a non-contact receiver 142 affixed thereto. The non-contact receiver 142 can be configured to detect the presence of the passive emitter 140 and output a signal representative thereof.

Non-contact receiver 142 can be included in order to detect the presence of the passive emitter 140. In some examples, passive emitter 140 comprises a magnet, and non-contact receiver 142 includes a magnet sensor configured to sense the presence of a magnetic field emitted from a magnetic passive emitter. In other examples, passive emitter 140 comprises a metallic component, and the non-contact receiver 142 comprises an inductive sensor. In some such embodiments, various system components, such as the spray arm assembly and/or stator portion can be made of plastic or other material that will not interfere with inductive detection of the passive emitter.

In still further embodiments, non-contact receiver can include an optical sensor configured to sense an optically detectable passive emitter. In some such examples, one or more components, such as some or all of the spray arm assembly and/or stator portion comprise a material transparent to one or more wavelengths used for the optical detection.

In still further embodiments, non-contact receiver can include a capacitive sensor configured to detect the presence of detectable passive emitter. In some such embodiments, one or more components, such as some or all of the spray arm assembly and/or stator portion comprise a material that will not affect or will minimally affect a capacitive coupling and/or electric field transmission between passive emitter and the non-contact receiver.

In the example shown, the stage 136 of the stator portion 134 is configured to cover the non-contact receiver 142 such that as the spray arm assembly 120 rotates, the passive emitter 140 traverses around one side of the stage 136 while the non-contact receiver is positioned on the opposite side of the stage. In some embodiments, rotation of the spray arm assembly 120 causes the passive emitter to move into and out of a detectable position proximate the non-contact receiver 142 once per revolution. Thus, in some such examples, as the spray arm assembly 120 is rotating, the non-contact receiver 142 can be configured to output a signal representing the presence of the passive emitter in the detectable position once per revolution. The resulting output signal from the non-contact receiver will be a series of pulses, with each pulse representing the completion of one rotation of the spray arm assembly 120.

Systems can include a controller in communication with the non-contact receiver and configured to receive a signal therefrom indicating the presence of a detectable emitter. In various examples, controller can include one or more processors, such as one or more microprocessors, digital signal processors (DSPs), application specific integrated circuits (ASICs), field programmable gate arrays (FPGAs), programmable logic circuitry, or the like, either alone or in any suitable combination. In some examples, the controller includes a processor and a memory configured to store software and data used or generated by the controller.

In some embodiments, the controller is configured to control one or more system components, such as one or more pumps and/or valves in order to provide fluid to the reprocessing system.

In some embodiments, the controller is in communication with the non-contact receiver 142 and is configured to detect when the passive emitter 140 is positioned at the detectable position proximate the non-contact receiver 142. In some examples, the controller receives a continuous or sampled signal from the non-contact receiver. The signal output from the non-contact receiver can be a steady value when the passive emitter is not within a detectable range of the non-contact receiver. The output of the non-contact receiver can change in response to the passive emitter being positioned at a detectable position. Such an output can include, for example, a detectable pulse.

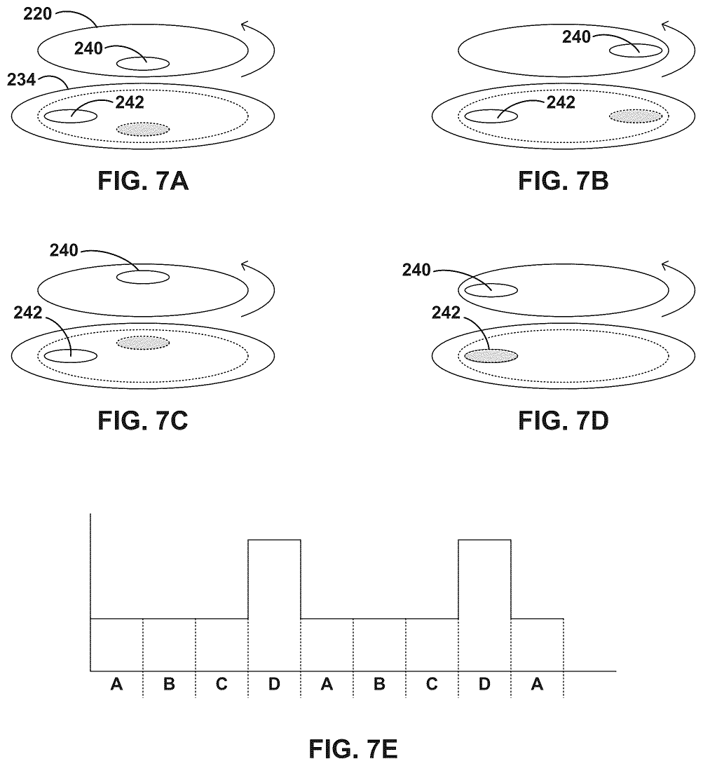

FIGS. 7A-FE show example operation of a non-contact receiver outputting a pulse signal representing the presence of a passive emitter within a detectable position. FIG. 7A shows a representation of spray arm assembly 220 including a passive emitter 240, such as a magnet embedded in a hub of a spray arm assembly. The spray arm assembly 220 is configured to rotation relative to a stator portion 234 of a processing chamber, such as a stage (e.g., stage 136). FIG. 7A shows an outline of the spray arm assembly 220 projected onto the stator portion 234 to show a relative alignment of the components according to the illustrated example as well as a shaded projection of the location of the passive emitter relative to the stator portion 234. A non-contact receiver 242, such as a magnet sensor, is supported by the stator portion 234. Though illustrated as being visible from the top of the stator portion 234, as described herein, in some examples, the non-contact receiver 242 is positioned below a surface of the stator portion 234, such as below a stage.

FIGS. 7A-FD show different rotational positions of the spray arm assembly 220 relative to the stator portion 234, shown by varying positions of the passive emitter 240 among the figures. in FIGS. 7A-FC, the passive emitter 240 is not aligned with the non-contact receiver 242, for example, as shown by the shaded location of a projection of the passive emitter onto the stator portion 234. However, in FIG. 7D, the passive emitter 240 is positioned generally above the non-contact receiver 242. In some examples, the non-contact receiver is configured to detect the presence of the passive emitter when positioned in a detectable position, which in some examples, is generally above the non-contact receiver, such as shown in FIG. 7D. In some embodiments, during operation, spray arm assembly 220 transitions in order between the rotational states shown in FIGS. 7A, 7B, 7C, and 7D and then repeats as the spray arm assembly 220 rotates.

FIG. 7E shows an example output from a non-contact receiver in response to rotation of a spray arm assembly and a passive emitter moving into and out of a detectable position. As shown in the example, an output signal is low when the passive emitter is not in a detectable position. For example, in the illustrated embodiment, the signal is low when passive emitter is in positions A, B, and C, corresponding generally to the orientation shown in FIGS. 7A, FB, and FC. The signal goes high when the passive emitter moves into a detectable position relative to the non-contact receiver, such as shown when passive emitter is in position D, corresponding generally to the orientation shown in FIG. 7C. As shown in the illustrated example, the signal from the non-contact receiver can be used to detect each time the passive emitter travels through a detectable position with respect to the non-contact receiver.

While shown as being a high signal when the passive emitter travels through the detectable position, in other examples, a signal might be high in times other than when the passive emitter travels through the detectable position. In some such examples, the signal output from the non-contact receiver goes low when the passive emitter is within the detectable region. In either case, the output of the non-contact receiver detectably changes when the passive emitter moves through a detectable position compared to when the passive emitter is not within a detectable position. This can result in a signal comprising pulses in the signal each time the passive emitter moves through the detectable position of the non-contact receiver. In some embodiments having a single passive emitter and a single non-contact receiver, each pulse generally corresponds to a single revolution of the spray arm assembly. Multiple passive emitters and/or multiple non-contact receivers can result in multiple pulses per revolution. Known numbers of passive emitters and/or non-contact receives can be used to determine how many pulse correspond to a single revolution of the spray arm assembly.

In some examples, the controller is configured to, each time the passive emitter is positioned at the detectable position proximate the non-contact receiver, increment a count of detected rotations of the spray arm assembly relative to the processing chamber. Thus, in some examples, the controller can maintain a count of the rotations of the spray arm assembly. In some embodiments, the controller is further configured to compare the count of detected rotations to an expected number of detected rotations. As described elsewhere herein, in some cases, a spray arm assembly does not rotate as expected in the event of fluid not being administered to the processing chamber as expected via the spray arm assembly. For instance, a rotation count lower than an expected rotation count may indicate one or more clogged fluid holes, an improperly coupled spray arm assembly, a malfunctioning fluid pump, a physical obstruction to rotation of the spray arm assembly, or some combination thereof. In some examples, if the controller determines that the count of detected rotations is below the expected number of detected rotations, the controller can output an alert. Alerts can include a visual display alert, such as illuminating one or more lights or outputting a graphical alert on a graphical display. Additionally or alternatively, an alert can include an audible alert. In some examples, an alert can be a networked alert, such as an alert via email, text message, or other network communication. Additionally or alternatively, in some examples, the controller can stop operation of the reprocessing system to prevent unsatisfactory reprocessing when the system is not operating in a normal state.

In some examples, the controller monitors a count of detected rotations of the spray arm assembly over time and can determine, for example, a rate of rotation of the spray arm assembly. In some such examples, comparing the count of detected rotations of the spray arm assembly to an expected number of detected rotations can include comparing a rate of rotation of the spray arm assembly to an expected rate of rotation. In some examples, the expected rate of rotation can be between 250 rpm and 600 rmp. In some examples, the expected rate of rotation can be approximately 300 rmp, approximately 480 rmp, or approximately 570 rpm. In some embodiments, the expected rate of rotation can depend on the configuration of the spray arm assembly, such as based on the number, size, and/or shape of spray holes. In some examples, the expected rate of rotation comprises a single value setting a minimum rate of rotation threshold. In other examples, the expected rate of rotation comprises a range of rates of rotations considered to be acceptable or within a normal range of rates of rotation.

In some embodiments, comparing the count of detected rotations to an expected number of detected rotations comprises comparing the count to a single threshold value, such as a minimum number of expected counts. In other examples, comparing the count of detected rotations to an expected number of detected rotations comprises comparing the count to a range of acceptable values, and the controller can be configured to output an alert and/or shut down the system to prevent further operation if the count is outside of the range. In some examples, comparing the count of detected rotations to an expected number of detected rotations comprises comparing the count to a single expected value, and the controller can be configured to output an alert and/or shut down the system to prevent further operation if the detected rotations deviates from the single expected value.

Confirming that the spray arm assembly is rotating at an expected speed can confirm that spray arm assembly is properly in place and is operating in the expected manner, which can be predetermined to provide an effective reprocessing (e.g., cleaning and/or sanitizing) process. In some examples, such confirmation confirms that fluid is provided at an expected volume and rate to expected locations, and that the rotational speed of the spray arm assembly is as expected.

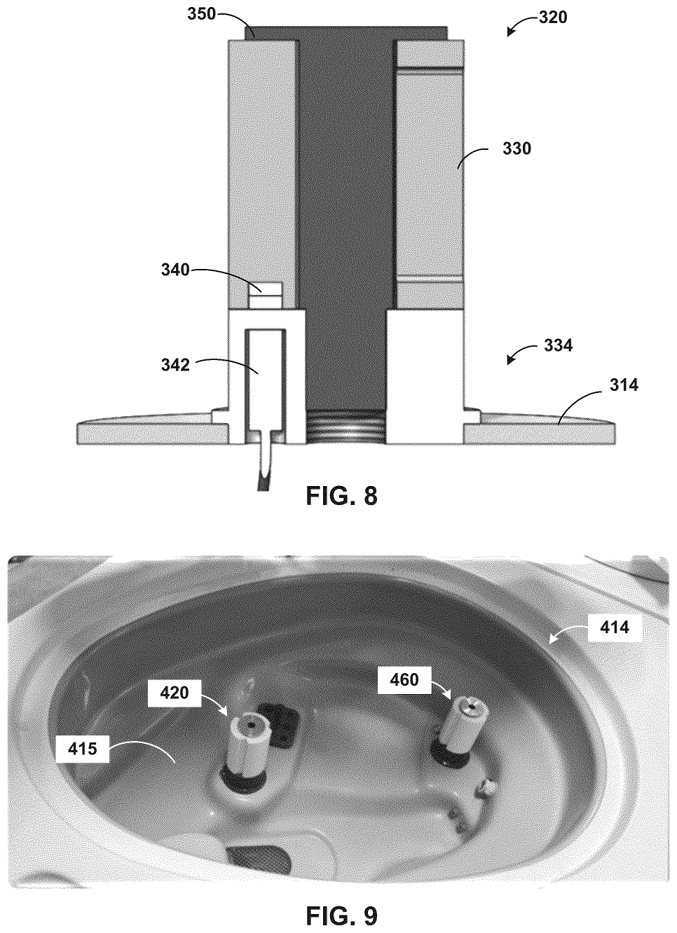

While various embodiments have been shown and described herein including spray arms generally parallel to a processing chamber floor, other configurations are possible within the scope of this disclosure. FIG. 8 shows a cross-sectional view of an example embodiment including a spray arm assembly including a spray arm extending parallel to a rotational axis and normal to the processing chamber floor.

In the illustrated example, spray arm assembly 320 coupled to a stator portion 334, such as a stator portion coupled to a floor of a processing chamber. The stator portion 334 includes a non-contact receiver 342 therein. Spray arm assembly 320 includes a spray arm 330 extending in a direction normal to a floor 315 of a processing chamber. In particular, in this example, spray arm 330 extends vertically rather than extending horizontally relative to the base or floor of the processing chamber. In some examples, spray arm 330 is configured to rotate relative to the stator portion 334 of the processing chamber 314. In some examples, spray arm assembly 320 is configured to rotate and expel fluid via one or more spray holes when a fluid is provided thereto from the processing chamber 314.

Spray arm assembly includes a passive emitter 340 configured to revolve around the rotational axis of the spray arm assembly 320 as it rotates. During rotation of the spray arm assembly 320, the passive emitter moves into and out of a detectable position relative to the non-contact receiver 342 such that it is positioned in the detectable position once per revolution of the spray arm assembly 320. A controller can be in communication with the non-contact receiver 342 such as described herein to monitor behavior of the spray arm assembly 320 during operation.

In the illustrated example, a cap 350 is positioned atop the spray arm assembly 320 and can be configured to remain fixed relative to the processing chamber 314 while the spray arm assembly 320 rotates. The cap 350 can include a spray hole therein such that fluid is applied into a cavity of the processing chamber 314 via both the spray hole(s) of the spray arm assembly 320 and the cap 350.

FIG. 9 shows a perspective view of an example reprocessing system including a processing chamber and a plurality of spray arm assemblies. As shown, processing chamber 414 includes a floor 415 having a first spray arm assembly 420 and a second spray arm assembly 460 extending therefrom. Spray arm assemblies can be configured similar to that shown in FIG. G.

In some examples, the plurality of spray arm assemblies can receive fluid simultaneously from the same fluid source. In other examples, separate fluid sources can individually provide fluid to respective spray arm assemblies. In various such examples, a system controller can control the application of one or more fluids to one or more corresponding spray arm assemblies. The controller can receive a signal from a non-contact receiver associated with each of the spray arm assemblies and monitor the number of detected rotations of the spray arm assembly such as described herein. The controller can determine whether each of the spray arm assemblies is operating as expected by comparing a detected number of rotations to an expected count and output an alert and/or shut down the system to prevent further operation if the detected number of rotations is not as expected such as described elsewhere herein.

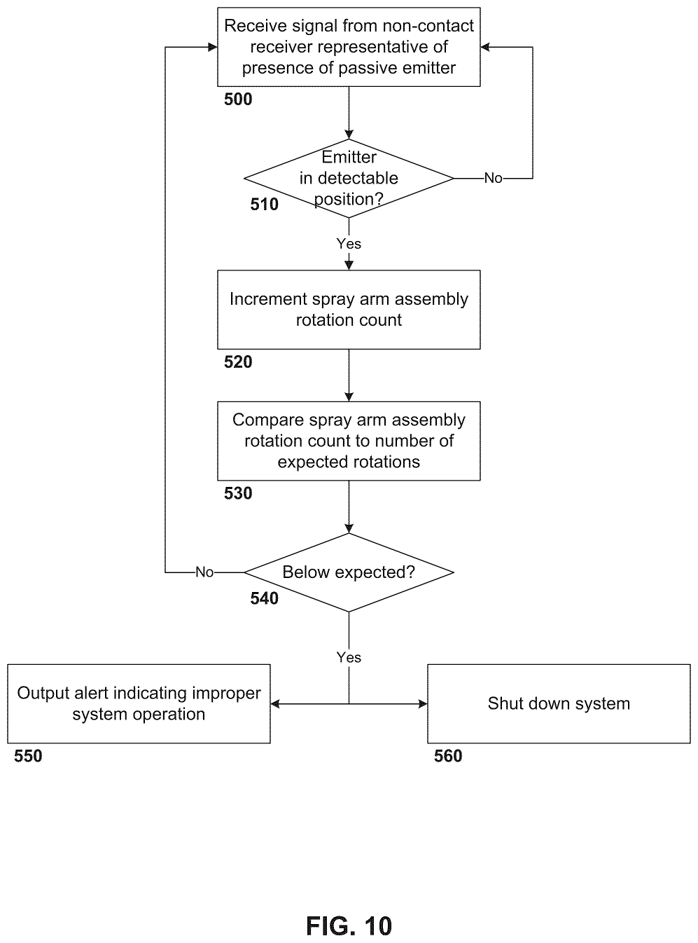

FIG. 10 is a process flow diagram showing an example operation of a controller determining whether the system is operating properly. In the illustrated example, the controller receives a signal from a non-contact receiver representative of a passive emitter (500). For example, the controller can receive a signal from a magnet sensor representative of whether the magnet is in a detectable position. In some examples, a detectable position comprises a position sufficiently close to the magnet sensor such that the magnet sensor senses a magnetic field from the magnet surpassing a threshold value. The controller can determine whether the emitter is in the detectable position (510), for example, by comparing a signal received from the magnet sensor to a detection threshold. If not, operation continues, and the process repeats at step 500. If the emitter is in the detectable position, the controller increments a spray arm assembly rotation count (520) and compares the spray arm assembly rotation count to a number of expected rotations (530). The controller can determine if the count is below an expected count (540), for example, by determining if the count is below a minimum expected number of counts for proper operation. If not, operation continues, and the process repeats at step 500. If the controller determines that the count is less than an expected count at step 540, the controller can output an alert (550). An alert can be an audible alert, a visual alert, a networked alert, or any combination thereof. Additionally or alternatively, in some examples, the controller can automatically shut down the system 560 and stop operation if the count is below the expected count to prevent a completion of an insufficient reprocessing procedure.

Various non-limiting examples have been described. These and others are within the scope of the following claims.

1. A medical device reprocessing system comprising:
a processing chamber configured to receive one or more medical devices to be cleaned;
a non-contact receiver coupled to the processing chamber, the non-contact receiver configured to detect a presence of a passive emitter; and
a spray arm assembly configured to rotate about a rotational axis relative to the processing chamber, the spray arm assembly comprising:
a spray arm configured to expel a fluid outward from the spray arm toward one or more medical devices within the processing chamber; and
the passive emitter configured to be detectable by the non-contact receiver, the passive emitter being positioned offset from the rotational axis such that rotation of the spray arm assembly relative to the processing chamber causes the passive emitter to move relative to the non-contact receiver.

2. The system of claim 1, wherein the passive emitter and the non-contact receiver are arranged such that, as the spray arm assembly rotates relative to the processing chamber, the passive emitter moves relative to the non-contact receiver such that the passive emitter moves to a detectable position proximate the non-contact receiver once per revolution of the spray arm assembly.

3. The system claim 1, further comprising a controller in communication with the non-contact receiver, wherein the controller is configured to:
detect when the passive emitter is positioned at a detectable position proximate the non-contact receiver; and
each time the passive emitter is positioned at the detectable position proximate the non-contact receiver, increment a count of detected rotations of the spray arm assembly relative to the processing chamber.

4. The system of claim 3, wherein the controller is configured to:
compare the count of detected rotations of the spray arm assembly relative to the processing chamber to an expected number of detected rotations; and
if the count of detected rotations is below the expected number of detected rotations, output an alert.

5. The system of claim 3, wherein the controller is configured to:
compare the count of detected rotations of the spray arm assembly relative to the processing chamber to an expected number of detected rotations; and
if the count of detected rotations is below the expected number of detected rotations, stop a reprocessing procedure.

6. The system of claim 1, wherein the processing chamber is configured to hold one or more endoscopes.

7. The system of claim 1, wherein the spray arm assembly is removably attachable to the processing chamber.

8. The system of claim 1, wherein the passive emitter comprises a magnet, and wherein the non-contact receiver comprises a magnet sensor.

9. The system of claim 1, wherein the spray arm assembly comprises a plurality of spray arms, each of the plurality of spray arms is configured to spray fluid outward from a respective one of the plurality of spray arms toward the one or more medical devices within the processing chamber, when the spray arm assembly is coupled to the processing chamber.

10. The system of claim 1, further comprising a cap configured to secure the spray arm assembly to the processing chamber, the cap being secured to the processing chamber such that the cap does not rotate when the spray arm assembly rotates relative to the processing chamber, and wherein the cap comprises a nozzle configured to emit fluid into the processing chamber.

11. The system of claim 1, wherein:
the processing chamber comprises a floor; and
the spray arm assembly is coupled to the floor of the processing chamber and the rotational axis is perpendicular to the floor of the processing chamber.

12. The system of claim 11, wherein the spray arm assembly comprises:
a first spray arm extending parallel to the floor of the processing chamber; and
a second spray arm extending parallel to the floor of the processing chamber and in an opposite direction as the first spray arm.

13. The system claim 11, wherein:
the first spray arm and the second spray arm extend along a spray arm axis;
the first spray arm comprises a first plurality of spray holes configured to receive fluid from the processing chamber, the first plurality of spray holes are separated from one another along the direction of the spray arm axis and are positioned on a first side of the spray arm axis;
the second spray arm comprises a second plurality of spray holes configured to receive fluid from the processing chamber, the second plurality of spray holes are separated from one another along the direction of the spray arm axis and are positioned on a second side of the spray arm axis, opposite the first, such that:
fluid spraying from the first plurality of spray holes imparts a first rotational force on the spray arm assembly to cause the spray arm assembly to rotate in a first direction; and
fluid spraying from the second plurality of spray holes imparts second rotational force on the spray arm assembly to cause the spray arm assembly to rotate in the first direction.

14. The system of claim 1, wherein the spray arm extends along the rotational axis and comprises a plurality of spray holes separated along a direction of the rotational axis.

15. The system of claim 1, wherein:
the processing chamber comprises a stator portion configured to engage the spray arm assembly and the spray arm assembly is configured to rotate relative to the stator portion; and
the non-contact receiver is held by the stator portion.

16. The system of claim 1, wherein the spray arm assembly is configured to rotate in response to fluid received from the processing chamber such that a rate of rotation of the spray arm assembly is related to a flow rate of fluid from the processing chamber and a flow rate of the fluid expelled from the spray arm.

17. A method of detecting proper connection of a rotating spray arm assembly in a medical device processing chamber, the method comprising:
delivering fluid to a spray arm assembly in a processing chamber configured to receive one or more medical devices, thereby causing the spray arm assembly to rotate;
detecting, by a non-contact receiver associated with the processing chamber, rotation of a passive emitter carried by the spray arm assembly as the spray arm assembly rotates to provide a detected number of rotations of the spray arm assembly;
comparing, by one or more processors, the detected number of rotations of the spray arm assembly to an expected number of rotations of the spray arm assembly; and
determining, by the one or more processors, that the spray arm assembly is improperly connected to the processing chamber if the detected number of rotations of the spray arm assembly is less than the expected number of rotations of the spray arm assembly.

18. The method of claim 17, wherein the passive emitter comprises a magnet and the non-contact receiver comprises a magnet sensor.

19. The method of claim 17, further comprising, in response to determining that the spray arm assembly is improperly connected to the processing chamber, issuing an alert.

20. The method of claim 17, further comprising, in response to determining that the spray arm assembly is improperly connected to the processing chamber, stopping delivery of fluid to the spray arm assembly.

21. The method of claim 17, wherein the processing chamber is part of an endoscope reprocessing machine, and the one or more medical devices comprises one or more endoscopes.

22. The method of claim 17, further comprising, prior to delivering fluid to the spray arm assembly, operatively connecting the spray arm assembly to the processing chamber.

23. The method of claim 17, further comprising determining, by the one or more processors, that the spray arm assembly is properly connected to the processing chamber if the detected number of rotations of the spray arm assembly is equal to or greater than the expected number of rotations of the spray arm assembly.
